(19) United States
(12) Patent Application Publication
(10) Pub. No.: US 2025/0001028 A1
(43) Pub. Date: Jan. 2, 2025

(54) Device for the vaporization of a sterilizing agent

(71) Applicant: DE LAMA S.P.A., San Martino Siccomario (IT)

(72) Inventor: Andrea Muggiasca, Pavia (IT)

(21) Appl. No.: 18/703,112

(22) PCT Filed: Oct. 28, 2022
(86) PCT No.: PCT/IB2022/060367

§ 371 Date: Apr. 19, 2024

(30) Foreign Application Priority Data
Oct. 28, 2021 (IT) 102021000027644

(51) Int. Cl. A61L 2/20; A61L 2/26; A61L 101/02

(52) U.S. Cl. CPC A61L 2/208; A61L 2/26; A61L 2101/02; A61L 2202/11; A61L 2202/121; A61L 2202/122; A61L 2202/14; A61L 2202/15

(57) ABSTRACT

A device generates (1) sterilizing vapor from liquid hydrogen peroxide fed into a sterilization chamber (CS), and includes: a metal vaporization pre-chamber (2) having a tubular shape and communicating with the sterilization chamber (CS); injection means (3) at the inlet of the vaporization pre-chamber (2) for feeding therein the liquid hydrogen peroxide, so that it is nebulized into particles about 40 μm in size or less; a helical induction coil coaxial to the outside of the vaporization pre-chamber (2) for heating of the thereof with consequent transition from the liquid to the vapor state of the hydrogen peroxide injected therein, with the electromagnetic field generated by the induction coil (4), in combination with the particle diameter of the nebulized particles acting to ionize the same particles to accelerate their aforementioned change of state from liquid to vapor.

### TECHNICAL FIELD

The present invention relates to the technical field concerning equipment for sterilizing or sanitizing products or environments by means of a sanitizing/sterilizing agent brought to a vapor state.

### BACKGROUND ART

Various vapor-generating devices of the type mentioned above are known, which are variously structured to suit specific applications and fields of use; depending on the requirements, devices are available that use elemental sanitizing agents, e.g., water steam, or other more valuable ones with characteristics that ensure safe sterilization and thus biological decontamination.

Among these sanitizing or sterilizing agents, one of the most widely used is hydrogen peroxide, which from its initial liquid condition is heated and brought to a vapor state, then fed into the chamber of a sterilization equipment containing products to be sterilized. A particularly preferred industrial field, though not the only one, for the use of such equipment is that relating to the packaging of pharmaceutical products in bottles or vials, which require their prior and complete sterilization before the drugs are introduced.

In this context, the preference given to hydrogen peroxide is due to its inherent characteristics, whereby it does not release toxic residual substances on the surfaces of the rooms and/or objects with which it came into contact during the sterilization process.

Since peroxide performs its sterilization functions exclusively by contact, it is on the other hand necessary to ensure that the entire surface, both external and internal, of the objects to be sterilized is reached by the sterilizing agent, and that it remains there for a sufficient time.

For this reason, peroxide is used almost exclusively by vapor-phase diffusion, rather than in liquid mixtures, whether nebulized or not, which would make it uncontrollable to actually achieve the targets.

Granted that it would be hard to count even partially the prior art devices that can be included in the same technical field as the present invention, and consequently to carry out an examination of their specific drawbacks, it is necessary to point out that the result that is obtained in a sterilization process depends, under the same conditions, on the time in which the vapor of the sanitizing agent used in the sterilization chamber remains in contact with the products to be sterilized, and therefore on minimum times of contact of the sanitizing agent with the products.

With known sterilization methods, in fact, the process of diffusing the vapor into the sterilization chamber, and covering the surfaces to be sterilized, requires even rather long times, which poorly matches the high productivity requirements of the packaging lines operating downstream of the sterilization equipment.

By way of example, a conventional vapor generator, the design of which has been widely known and used in the sterilization of pharmaceutical containers for several years, comprises a roughly cylindrical body, closed at one end and open at the opposite end, at which it is connected with the sterilization chamber of a sterilization equipment.

The generator defines, within itself, a vaporization chamber, in which a plate-shaped electric heating device is provided. A special pipeline allows a metered stream of sterilizing agent, usually 30%-35% hydrogen peroxide taken from a reservoir outside the generator, to be dripped by gravity onto the heated plate of the heating device.

Vaporization of the peroxide therefore occurs by contact with the superheated surface of the plate. The vapor thus generated is then transferred to the sterilization chamber. The transfer can take place by natural diffusion, by virtue of the overpressure created in the vaporization chamber relative to the sterilization chamber as a result of vapor formation, or by supplying to the vaporization chamber air taken from outside and previously filtered to prevent contaminants from entering the sterilization equipment.

A suitably programmed control unit, which continuously detects temperature and pressure values within the vaporization chamber and, if necessary, within the sterilization chamber, adjusts the vapor production to ensure that the desired amount of vaporized peroxide reaches the sterilization chamber and the components to be sterilized according to preset sterilization programs.

Such programs usually involve a number of cycles of vapor production and introduction into the sterilization chamber, alternating with settling periods, during which the vapor produced diffuses into the sterilization chamber and, with times depending on the vapor flow rate, its temperature and the turbulence in the sterilization chamber itself, reaches all surfaces, external and internal, of the objects to be sterilized. Some of the known vapor generators are isolated from the external environment, are pressure-tight and thus operate under partial vacuum conditions (typically greater than 10 mbar). In this case, diffusion of peroxide vapor occurs solely due to the overpressure generated by the formation of the vapor itself.

### Technical Problem

The generators described above are not particularly efficient in producing peroxide vapor, and in addition, obtaining acceptable sterile conditions requires a number of production and settling cycles, typically at least four or five cycles. All of this makes the stay time of the objects inside the sterilization equipment quite long.

### OBJECTS OF THE INVENTION

Therefore, the object of the present invention is to propose a device for generating vapor of a liquid sterilizing or sanitizing agent, which allows to improve the vaporization efficiency of the same in comparison with vaporizing devices of the prior art.

Another object of the invention is to propose a device capable of improving the time for covering the surfaces to be sterilized currently achievable by devices of the prior art.

Still another object of the invention is to come up with a device that is simple, though not obvious, so that this simplicity of construction is a guarantee of reliable operation and consistent results.

A further object of the invention is to propose a device to be associated with a sterilization chamber brought to a depressurized condition, sealed off from the external environment and provided, for example, to contain objects to be decontaminated.

Still another object of the invention relates to the proposal of a device which preferably uses, as a sanitizing or sterilizing agent, hydrogen peroxide, but which may, if necessary, use another type of originally liquid agent that is deemed more suitable for a particular application.

### SUMMARY OF THE INVENTION

These and other objects are fully achieved by means of a device for generating sterilizing vapor from a liquid sanitizing or sterilizing agent, for use in a sterilization chamber brought to a depressurized condition and sealed off from the external environment.

The above mentioned device comprises:
a vaporization pre-chamber, having a tubular shape and made communicating with said sterilization chamber;
injection means, arranged at the inlet of said tubular vaporization pre-chamber and provided for feeding said liquid sanitizing or sterilizing agent into said chamber, so that the agent is nebulized into particles having a diameter between 5 and 50 micrometers;
at least one induction coil, having a helical shape, arranged coaxially outside said tubular vaporization pre-chamber and extended axially for a predetermined portion of the total length of said tubular vaporization pre-chamber, the coil being intended for the heating of the pre-chamber with consequent transition from the liquid to the vapor state of said sanitizing or sterilizing agent injected therein, with an electromagnetic field generated by said induction coil, in combination with the intended particle diameter of the sanitizing or sterilizing agent, intended to ionize the same particles to accelerate their aforementioned change of state from liquid to vapor.

### BRIEF DESCRIPTION OF THE DRAWINGS

The features of the invention will be apparent from the following description of a preferred embodiment of the device for generating sterilizing vapor from a liquid sanitizing or sterilizing agent herein described, in accordance with what has been proposed in the claims and with help of the enclosed figures, in which:

FIG. 1 illustrates an axonometric view of a preferred embodiment of the vapor generating device herein described;

FIG. 2 illustrates a view similar to FIG. 1 with parts removed to better show others:

FIG. 3 illustrates an axial section of the device;

FIG. 4 illustrates a schematic, block view of a plant in which the device is associated in order to serve a sterilization chamber.

### DESCRIPTION OF EMBODIMENTS OF THE INVENTION

In the above figures, the device for vaporizing a sterilizing agent made according to an embodiment of the invention has been indicated by reference 1 as a whole. The vaporizer device 1 is intended to operate in a sterilization apparatus comprising a sterilization chamber CS created within an enclosed body of said sterilization equipment.

The device 1 provides for the generation of sterilizing vapor from a liquid sanitizing or sterilizing agent, preferably, though not necessarily, consisting of hydrogen peroxide. In the following, for sake of simplicity, reference will be made to the use of hydrogen peroxide as the sterilizing agent, although this is not a limiting factor in the scope of the invention.

Sterilizing vapor is used, for example, inside the aforementioned sterilization chamber CS, set under vacuum by vacuum generating means V and suitably sealed off from the external environment during the operation of the device 1.

In a manner per se known, the sterilization chamber CS is provided with a duct K equipped with a shut-off valve W, which can be opened on command to connect the external environment with the interior of the sterilization chamber CS to restore atmospheric pressure in the latter.

In the embodiment shown herein, the device 1 comprises a substantially cylindrical body 1a, closed at one end, defining within it a vaporization pre-chamber 2, having a tubular shape and made communicating with said sterilization chamber CS.

The body 1a defining the vaporization pre-chamber 2 is made of metal which, for the most critical applications, is preferably stainless steel. The mechanical characteristics of the body 1a are sized to make it tight against pressurization and depressurization, so that it can guarantee perfect operation under partial vacuum conditions, typically but not exclusively in the range of 2 to 5 mbar abs.

As an example, to highlight empirically optimized size ratios, the body 1a may have:
an internal diameter of about 60 mm;
a wall thickness of about 1.65 mm;
an overall length of about 180 mm.

An inlet head 20 of the tubular vaporization pre-chamber 2 is formed at the closed end of the body 1a; injection means 3, provided for injecting finely nebulized hydrogen peroxide inside the same tubular vaporization pre-chamber 2, are attached to the inlet head 20; while the remaining outlet head 21 is open and communicates with the sterilization chamber CS.

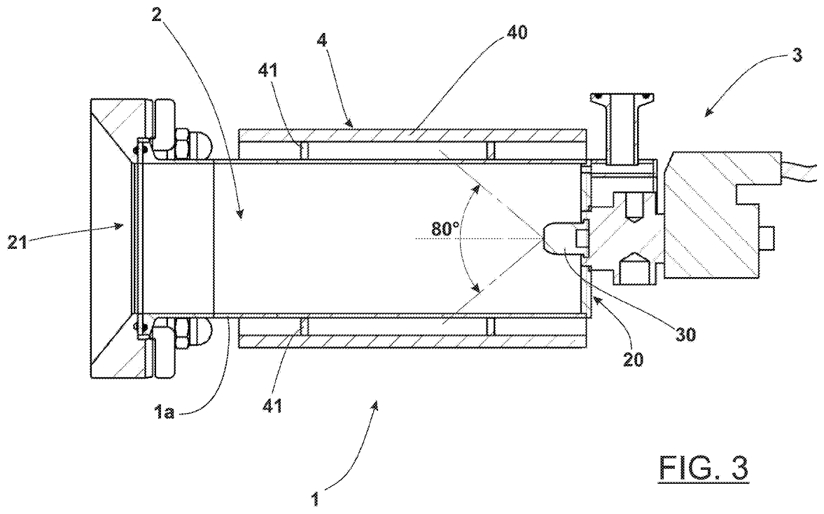

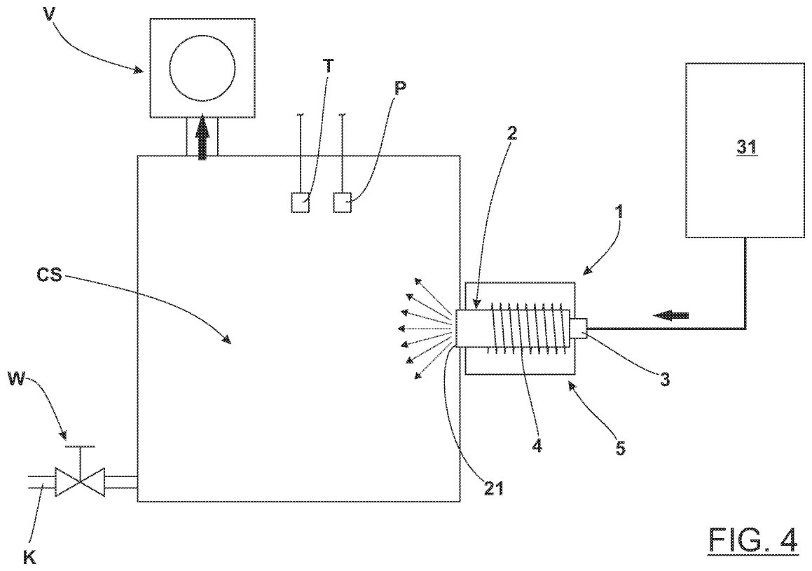

Communication between the tubular vaporization pre-chamber 2 and the sterilization chamber CS can be accomplished by direct entry into the latter of said outlet head 21 (FIG. 3) or by interposition of a connecting pipeline, not shown as being of obvious understanding.

In an alternative embodiment of device 1, that is not illustrated, the body 1a consists of a part of the sterilization chamber CS body, and extends outward as part of the same, as a single body. The functionalities are the same, but in terms of construction the assembly is easier to make and maintain and less prone to pressure loss.

The aforementioned injection means 3 have a nozzle 30, disposed centrally with respect to the tubular vaporization pre-chamber 2 and fed, by known means 31 not illustrated in detail, with liquid hydrogen peroxide at 30 volumes at a pressure of between 4 to 8 bar, and preferably about 6 bar, taken from a suitable tank also not illustrated.

Advantageously, for the objects of the invention, the nozzle 30 is adapted to generate a spray cone having preferably an amplitude between 70° to 90°, and preferably about 80°, in which the peroxide is nebulized in particles having a diameter of less than 50 micrometers, and preferably less than 40 micrometers and greater than 5 micrometers.

Coaxially arranged outside said tubular vaporization pre-chamber 2 is at least one induction coil 4, having a helical shape, extended axially for a predetermined portion of the total length of said tubular vaporization pre-chamber 2. The coil 4 is intended to generate a variable electromagnetic field within its turns which, by known effects of electromagnetic induction, generates a variable electric current in the metal body 1a which, by Joule effect, heats the body 1a itself.

Vaporization of the peroxide takes place primarily by the contact of the microscopic nebulized peroxide droplets with the inner wall of the body 1a, and secondarily in the vaporization chamber 2, as a result of the temperature rise that nonetheless occurs within it and the interaction of the droplets with the high-temperature vapor already formed. The very small size of the atomized droplets causes them to vaporize immediately upon first contact with the inner wall of body 1a.

The operation temperature of body 1a can be adjusted, either by changing the intensity of the current circulating in coil 4 or by defining appropriate on-off cycles of the same current.

The formation of peroxide vapor induces an overpressure in the vaporization chamber relative to that present in the sterilization chamber, so the vapor tends to diffuse naturally within the latter. Conveying within the sterilization chamber CS is also facilitated by the mechanical action of peroxide atomization carried out through the nozzle.

Roughly, in the dimensional example described above, the length of the induction coil 4 is 140 mm in relation to an overall length of about 180 mm of the tubular vaporization pre-chamber 2, starting from the aforementioned inlet head 20 of the latter.

Advantageously, the induction coil 4 turns out to be slightly spaced from the wall of the tubular vaporization pre-chamber 2, e.g., by providing a Teflon spool 40 suitable for supporting the winding of the said induction coil 4 and fixed, by means of spacers 41, to the outside of the same tubular vaporization pre-chamber 2.

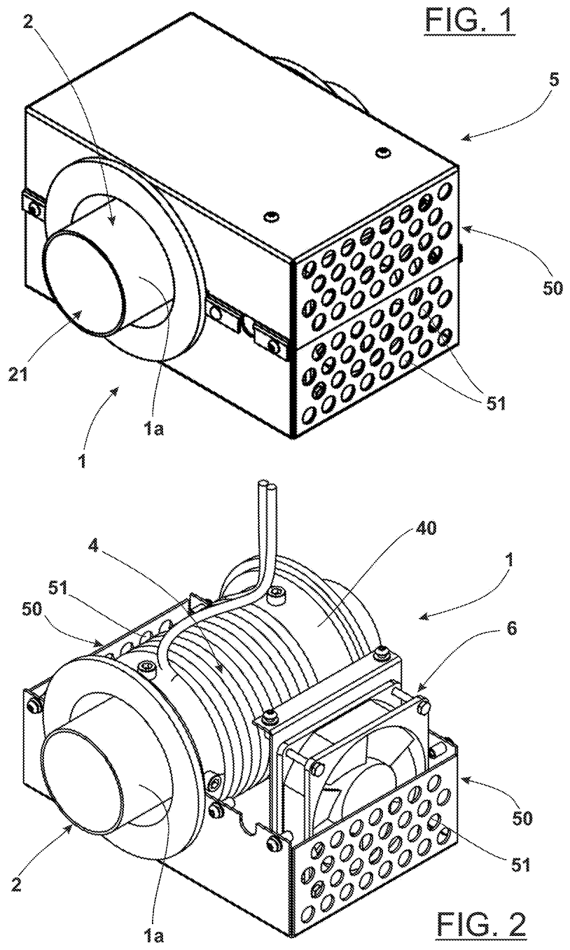

The assembly comprising the body 1a and the associated induction coil 4, in the preferred embodiment of the device shown in FIGS. 1 and 2, is advantageously housed within a box-like casing 5 having at least some of its walls 50 made permeable to air through holes 51 or ventilation grilles.

The box-like casing 5 is also preferably made of stainless steel sheet metal.

At least one fan 6 is provided within the box-like casing 5 for cooling the tubular vaporization pre-chamber 2 and induction coil 2.

A computerized and programmable control unit, not shown, is provided to define the quantities of peroxide vapor that are produced and the various modes of production. These modes, the same as with some known vapor generators, are generally divided into one or more cycles, each comprising a vapor generation phase and a settling phase, during which the vapor produced spreads into the sterilization chamber and affects the products to be sterilized.

The production parameters are controlled in a known manner by special temperature, pressure and vapor concentration sensors, provided, for example, inside the vaporing chamber and electrically connected to the control unit.

According to the invention, the particular configuration of the vaporization device 1 and its sizing, with particular reference to the size of the nebulized peroxide droplets inside the vaporization chamber 2 and the presence of the electromagnetic field generated by the induction coil 4, cause the peroxide inside the chamber 2 to undergo, in addition to heating, an ionization process, that makes electrically charged the nebulized liquid droplets first and then the peroxide vapor.

This fact results both in improved vaporization efficiency, because the charged peroxide droplets tend to adhere more readily to the inner wall of the body 1a, and in improved efficiency in the sterilization process, as the ionized vapor particles contact all surfaces of the products to be sterilized better and faster.

The values for the size of the nebulized peroxide droplets were derived experimentally, so as to optimize the ionization effect, which is a critical feature of the invention.

Hydrogen peroxide vapor is then drawn in by the relative vacuum present in the sterilization chamber CS and spreads into the latter, also coming in contact with the surfaces of the objects to be sterilized.

As previously mentioned, device 1 may provide temperature control means T, of a substantially known type, for controlling the temperature of the vapor of said hydrogen peroxide, for example but not necessarily, located in the sterilization chamber CS.

In addition or alternatively, pressure control means P may be provided for controlling the vapor pressure of hydrogen peroxide, located in the sterilization chamber CS.

In a first design variant, shut-off means, not shown as being of intuitive understanding, may be provided in device 1 interposed between said tubular vaporization pre-chamber 2 and sterilization chamber CS, operable to open or close the connection between them; such shut-off means may be advantageous as they can anticipate the onset of vapor transformation of hydrogen peroxide in the tubular vaporization pre-chamber 2 with respect to its entry into the sterilization chamber CS.

From the above description, the advantageous aspects that are obtained with the vapor generation device proposed by the present invention are clear and obvious.

In particular, attention is drawn to the technical/constructive arrangements of the device, which concur to obtain a sterilizing vapor, from hydrogen peroxide or similar liquid sanitizing/sterilizing agent, that possesses optimal physical characteristics for the function to be performed, so as to exceed, in performance, what can be obtained with the devices of the prior art.

Another advantageous aspect given by the device is the described molecular ionization transformation of hydrogen peroxide during its vaporization, which therefore enhances its decontaminating, sanitizing and sterilizing performance.

The proposed device, as is also evident from observation of the attached drawings, is constructively very simple, with no moving and/or wearing parts, ensuring reliable operation and consistent results over time.

An additional advantage derived from the invention is that, within the sterilization chamber CS, vaporized and ionized hydrogen peroxide (or other oxidizing agent) is capable of sterilizing even objects enclosed in sealed pouches made entirely or partially of Tyvek and/or medical paper.

As already stated in the description, the device is intended to use preferably hydrogen peroxide as a sanitizing or sterilizing agent, but the same device is also suitable to use, if necessary, another type of originally liquid agent that is deemed more suitable for a particular application.

Anyway, it is understood that what is described above is illustrative and not limiting, therefore any detail variations that may be necessary for technical and/or functional reasons are to be considered from now on within the same protection scope defined by the claims below.

1. A device (1) for generating sterilizing vapor from a liquid sanitizing or sterilizing agent operable in a sterilization chamber (CS) of an equipment for sterilization of products when brought to a depressurized condition and sealed off from an external environment that is external to said sterilization chamber CS), said device (1) by comprising:
a body (1a), defining a vaporization pre-chamber (2) having a tubular shape and configured for communication with said sterilization chamber (CS);
injection means (3), arranged at an inlet of said vaporization pre-chamber (2) and provided for feeding said liquid sanitizing or sterilizing agent into said vaporization pre-chamber (2), said injection means (3) configured for nebulizing said liquid sanitizing or sterilizing agent to obtain nebulized particles having an average diameter of less than 50 micrometers;
at least one induction coil (4), arranged coaxially on an outside of said vaporization pre-chamber (2) and extended axially for a portion of a total length of said vaporization pre-chamber (2) and configured to bring about heating of said body (1a) including said pre-chamber (2) with an electromagnetic field generated by said induction coil (4) configured to produce ionization of said sanitizing or sterilizing agent, and wherein a temperature increase generated in said body (1a) and within said pre-chamber (2) is configured to vaporize said sanitizing or sterilizing agent injected therein with the diameter of the nebulized particles of the sanitizing or sterilizing agent configured to promote ionization of the nebulized particles and to accelerate their vaporization of the nebulized particles.

2. The device according to claim 1, wherein said vaporization pre-chamber (2) comprises an inlet head (20) for positioning said injection means (3) closed to the external environment, and wherein an outlet head (21) of said vaporization pre-chamber (2) is open for flow of vaporized particles from the vaporization pre-chamber (2) directly into said sterilization chamber (CS).

3. The device according to claim 1, wherein said vaporization pre-chamber (2) comprises an inlet head (20) for positioning said injection means (3) closed to the external environment, and a connecting pipeline is provided between a remaining open outlet head (21) of said vaporization pre-chamber (2) and said sterilization chamber (CS).

4. The device according to claim 1, wherein said vaporization pre-chamber (2) is made of a stainless steel tube having: an inner diameter of substantially 60 mm; a wall thickness of substantially 1.65 mm; an overall length of substantially 180 mm.

5. The device according to claim 1, wherein a nozzle (30) is provided in said injection means (3) and arranged in a central position with respect to said vaporization pre-chamber (2), and wherein said nozzle (30) is supplied with said liquid sanitizing or sterilizing agent at a pressure between 4 to 7 bars, and wherein said nozzle (30) is configured to generate a spray cone between 70° to 90° of said liquid sanitizing or sterilizing agent.

6. The device according to claim 5, wherein said nozzle is integrated into a solenoid valve (30) that is configured to control the injection of said liquid sanitizing or sterilizing agent.

7. The device according to claim 1, wherein said liquid sanitizing or sterilizing agent is nebulized into particles having an average diameter of 40 micrometers.

8. The device according to claim 1, wherein a spool (40) of insulating material is associated, by means of spacers (41), the outside of the vaporization pre-chamber (2) and configured to support a winding of said induction coil (4).

9. The device according to claim 1, further comprising a box-like casing (5) with walls (50) permeable to air for containing said tubular vaporization pre-chamber (2) and induction coil (4), and at least one fan (6) is installed within the box-like casing (5) for cooling said vaporization pre-chamber (2) and induction coil (4).

10. The device according to claim 1, further comprising temperature control means (T) for temperature control of said sanitizing or sterilizing agent in said sterilization chamber (CS).

11. The device according to claim 1, further comprising pressure control means (P) for pressure control of said sanitizing or sterilizing agent in said sterilization chamber (CS).

12. The device according to claim 1, further comprising shut-off means interposed between said vaporization pre-chamber (2) and said sterilization chamber (CS) operable to open or close a connection therebetween.

13. The device according to claim 1, wherein said liquid sanitizing or sterilizing agent consists of hydrogen peroxide.

14. The device according to claim 1, wherein said body (1a) is part of the sterilization chamber (CS) and extends outwardly therefrom as an extension thereof.
